(19) United States
(12) Patent Application Publication
(10) Pub. No.: US 2007/0060886 A1
(43) Pub. Date: Mar. 15, 2007

(54) Syringe

(76) Inventor: William R. Knepshield, Kennedyville, MD (US)

Correspondence Address: HOWSON AND HOWSON, SUITE 210, 501 OFFICE CENTER DRIVE, FT WASHINGTON, PA, 19034, US

(21) Appl. No.: 11/484,291

(22) Filed: Jul. 11, 2006

Related U.S. Application Data
(60) Provisional application No. 60/711,172, filed on Aug. 25, 2005.

(51) Int. Cl. A61M 5/00

(52) U.S. Cl. 604/110

(57) ABSTRACT

In a safety syringe in which a needle on a needle holder is withdrawn into the syringe barrel after use, the needle holder has resilient, proximally extending, fingers with radial projections which enter recesses formed in the interior wall of the barrel to lock the needle holder in place. Distally extending fingers on the plunger cooperate with the proximally extending fingers of the needle holder to release the projections from the recesses, and cooperating notches and projections on the fingers latch the needle holder to the plunger so that the needle holder, with the needle attached to it, can be withdrawn into the barrel.

### CROSS-REFERENCE TO RELATED APPLICATION

This application claims priority from provisional application 60/711,172, filed Aug. 25, 2005. The disclosure of provisional application 60/711,172 is hereby incorporated by reference in its entirety.

### FIELD OF THE INVENTION

This invention relates to safety syringes, and more particularly to disposable safety syringes of the type in which, following a single use, for example the administration of an injection, the needle of the syringe can be engaged by the plunger, and withdrawn into the interior of the syringe barrel, where it cannot cause injury.

### BACKGROUND OF THE INVENTION

A problem encountered in some safety syringes is that, in order to allow the needle to be withdrawn into the barrel, the needle hub fitting, that is the fitting to which the needle hub is attached, must be releasably connected to the distal end of the barrel. If the releasable connection between the needle hub fitting and the syringe barrel is too weak, the needle assembly can be pushed into the barrel in the process of inserting the needle through the stopper of a vial, or through the skin of a patient.

The syringe described in U.S. Pat. No. 6,821,266, dated Nov. 23, 2004, has a firm frictional connection between the needle hub fitting and the barrel, and utilizes a coupling device that establishes a positive connection between the plunger and the needle hub fitting to ensure that the friction holding the needle hub in place at the distal end of the barrel will be overcome by withdrawal of the plunger when coupling device is engaged. Because the syringe of Patent 6,821,266 relies on a firm frictional connection between the needle hub fitting and the barrel, a relatively high pulling force is required to dislodge the needle from the barrel.

U.S. Pat. No. 6,468,246, dated Oct. 22, 2002, and U.S. Pat. No. 6,488,657, dated Dec. 3, 2002, address the problem by avoiding reliance on friction, and instead provide releasable positive connection of the needle hub fitting to the syringe barrel.

The syringe of Patent No. 6,468,246 utilizes an expansion ring that normally holds spring catches on a needle hub fitting outward and in engagement with detent surfaces. In operation, the plunger is pushed forward to push the expansion ring forward and into a recess formed in the needle hub fitting. This releases the catches. Then, when the plunger is pulled back, it engages the inwardly deflected catches, which then serve a second purpose, allowing the plunger to pull the needle hub fitting backward, thereby withdrawing the needle into the barrel.

The syringe of Patent No. 6,488,657 utilizes a locating ring that normally holds catches on a needle hub fitting outward and in engagement with detents on the inside wall of the barrel. The plunger is pushed forward to catch the ring, and then pulled back to release the engagement of the catches with the detents. When the plunger is pulled back farther, a secondary catch on the ring engages a flange on the needle hub fitting to pull the needle hub fitting backward, thereby withdrawing the needle into the barrel.

### BRIEF SUMMARY OF THE INVENTION

An object of this invention is to avoid the problems associated with frictional engagement between a needle hub and a syringe barrel, and to provide a more simple, yet reliable, releasable positive connection between the needle hub and the barrel.

A preferred safety syringe according to the invention comprises, as its principal elements, a syringe barrel, a needle holder, and a plunger. The barrel has proximal and distal ends, a cylindrical internal wall, and a recess formed in the cylindrical internal wall adjacent the distal end of the barrel. The needle holder is fitted to the distal end of the barrel and connected to a needle extending distally away from the distal end of the barrel. The plunger is slidable in the barrel, and extends proximally from the barrel so that it can be engaged manually to effect sliding movement of the plunger. A stopper, fixed to the distal end of the plunger, is in slidable sealing engagement with the cylindrical internal wall of the barrel. A resilient finger is fixed to, and extends proximally from, the needle holder within the barrel and alongside the cylindrical wall of the barrel. The finger has a cam surface formed at its proximal end, and a radial projection extending into the recess formed in the internal wall of the barrel. The engagement of the radial projection with the recess positively secures the needle holder against distal movement within the barrel. Another finger is fixed to, and extends distally from, the plunger within the barrel and alongside the cylindrical wall of the barrel. The distally extending finger has a distal end engageable with the cam surface for urging the resilient finger radially inward to disengage its radial projection from the recess. One of the fingers has a radial projection, and a notch is formed in the other finger for receiving the projection. The projection and notch engageable with each other for latching the fingers together while the radial projection is disengaged from the recess in the wall of the barrel. Therefore, when the plunger is pushed toward the distal end of the barrel, it can disengage the needle holder from the barrel, and, upon reversal, it can withdraw the needle holder, with the needle, into the barrel.

Other objects, details and advantages of the invention will be apparent from the following detailed description when read in conjunction with the drawings.

### BRIEF DESCRIPTION OF THE DRAWINGS

FIG. 1 is an axial section of a syringe in accordance with the invention;

FIG. 2 is a fragmentary axial section showing, in greater detail, the initial stage of engagement of a plunger projection with a needle holder projection; and

FIG. 3 is a similar fragmentary axial section showing the final stage of engagement of the plunger projection with the needle holder projection.

### DETAILED DESCRIPTION OF THE PREFERRED EMBODIMENT

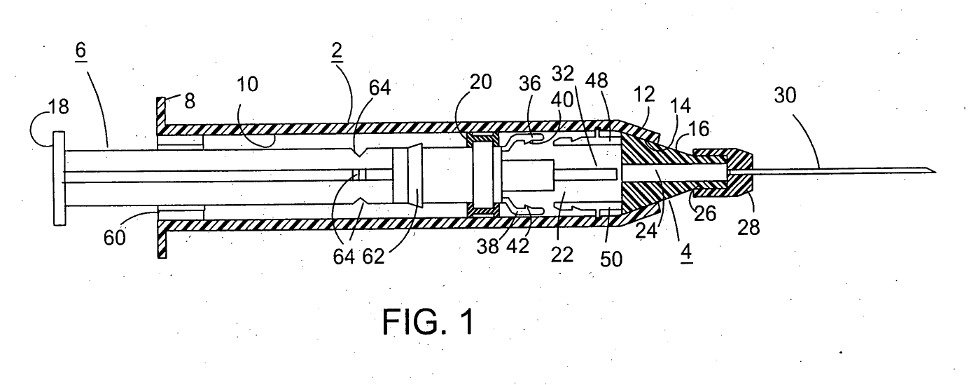

The principal elements of the syringe shown in FIG. 1 are the barrel 2, the needle holder 4 and the plunger 6. The barrel 2 is preferably composed of a transparent or semi-transparent synthetic resin, and has a flange 8 at its proximal end, a circular, cylindrical interior wall 10 throughout almost its entire length, and a tapered part 12 at its distal end, providing a frusto-conical internal surface 14 against which a mating external frusto-conical surface 16 of the needle holder 4 is in seating relationship.

The plunger 6 has a button 18 which is engageable by the user's thumb, while two fingers are engaged with flange 8, in order to depress the plunger. An elastomeric stopper 20 is provided on the plunger adjacent the distal end of the plunger, and is in slidable, sealing engagement with the cylindrical internal wall 10 of the barrel, thereby forming an enclosed fluid chamber 22, the only outlet of which is an internal passage 24 of the needle holder. An extension 26 of the needle holder projects through the distal end of the barrel, and is connected to the hub 28 of a needle assembly including the hub 28 and a needle cannula 30. The plunger 6 has a centrally located extension 32, which enters the internal passage 24 of the needle holder 4 for guidance when the plunger is depressed.

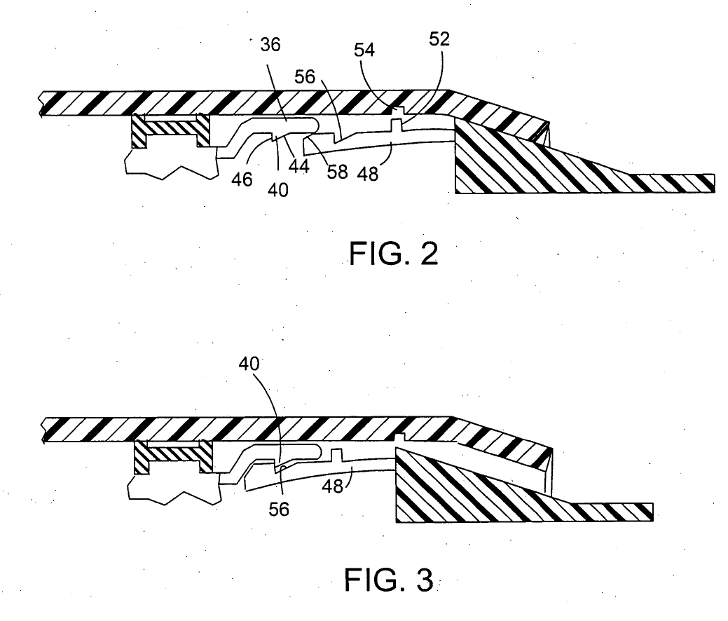

A pair of fingers 36 and 38 extend distally from the plunger and are disposed at diametrically opposite positions on the plunger. These fingers are preferably molded as a unit with the plunger, and are shaped so that their proximal ends meet the plunger at a sufficient distance radially inward from the wall 10 to allow space for the stopper 20. The major parts of the fingers 36 and 38 extend axially along wall 10 and are preferably in direct sliding contact with the wall 10, but can be in closely spaced relationship with the wall. These fingers have triangular-shaped projections 40 and 42, which extend radially inward. As shown in FIG. 2, projection 40 has a sloping distal face 44 and a radial or nearly radial proximal face 46.

The needle holder has a pair of diametrically opposite, resilient fingers 48 and 50. These fingers are preferably molded as a unit with the needle holder. They extend proximally from the needle holder. Finger 48 has a projection 52, which extends radially outward, and into a recess 54 formed in the inner wall of the barrel. The recess 54 is seen in FIGS. 2 and 3. Finger 50 has a similar projection, which also extends into a recess formed in the inner wall of the barrel. The recesses can be separate recesses, or parts of a single annular groove. Fingers 48 and 50 have triangular notches shaped to mate with the triangular projections on fingers 36 and 38, the notch 56 in finger 48 being shown in FIG. 2.

As shown in FIG. 2, as the plunger is pushed in the distal direction, the distal end of finger 36 engages a cam surface 58 at the proximal end of finger 48, causing finger 48 to bend inward so that projection 52 disengages recess 54. As shown in FIG. 3, when the plunger is pushed still farther in the distal direction, the projection 40 enters notch 56, and radial face 46 on projection 40 comes into engagement with a nearly radial face of notch 56. A similar action occurs when finger 38 engages finger 50. The resilient fingers 48 and 50 snap outward when the projections of fingers 35 and 38 engage the notches of fingers 48 and 50. However, the thicknesses of fingers 36 and 38 are such that the radial outward projection, e.g. projection 52 on finger 48, do not reenter the recesses in the wall of the barrel. Consequently the needle holder can be unseated from the frusto-conical surface 14 at the distal end of the barrel, and pulled proximally, by pulling on the plunger to withdraw the needle assembly into the interior of the barrel.

An insert 60, depicted schematically in FIG. 1 cooperates with a cam-shaped annulus 62 formed on the plunger, to prevent the plunger from being pushed inward after the needle is withdrawn into the interior of the barrel. For details of the insert and the manner in which it cooperates with the cam-shaped annulus, reference may be made to U.S. Pat. No. 6,821,266, the entire disclosure of which is here incorporated by reference.

The cooperating fingers on the needle holder ensure that the needle holder is positively secured to the distal end of the barrel so that it cannot be pushed away from its seat and into the barrel as the needle is being accidentally pushed into a vial, or as an injection is being administered. The cooperating fingers of the plunger and needle holder, however, make it possible to release the needle holder by pushing on the plunger, and to withdraw the needle into the barrel easily by pulling on the plunger. The plunger and the needle holder can be readily produced by molding, and assembly of the syringe is simple, involving only a minimum number of parts.

Various modifications can be made to the syringe as described above. For example, the needle assembly and the needle holder need not be separate, detachable elements. The needle cannula can be permanently connected to the needle holder, eliminating the separate hub 28. The number of fingers on the plunger preferably matches the number of fingers on the needle holder. However the number is not critical. The plunger and needle holder can have one finger each, or each may have more than two fingers. Preferably, two or more fingers are provided on the plunger and on the needle holder, and the fingers are arranged symmetrically about the axis of the barrel so that the lateral forces exerted by the fingers balance one another. Although the triangular notches are preferably in the fingers on the needle holder, and the triangular projections are preferably on the fingers of the plunger, as an alternative, the notches can be on the fingers of the plunger, and the projections can be on the fingers of the needle holder. Moreover, the projections and notches, while desirably triangular, can be formed in various other shapes and still function in substantially the same way.

Still other modifications may be made to the apparatus and method described above without departing from the scope of the invention as defined in the following claims.

I claim:

1. A safety syringe comprising:
a syringe barrel having proximal and distal ends, a cylindrical internal wall, and a recess formed in the cylindrical internal wall adjacent the distal end of the barrel;
a needle holder fitted to the distal end of the barrel and connected to a needle extending distally away from the distal end of the barrel;
a plunger slidable in the barrel, the plunger also having proximal and distal ends and a stopper fixed to the distal end of the plunger and in slidable sealing engagement with the cylindrical internal wall of the barrel, the plunger extending proximally from the barrel and engageable manually to effect sliding movement of the plunger;
a resilient finger fixed to, and extending proximally from, the needle holder within the barrel and alongside the cylindrical wall, the resilient finger having a proximal end, a cam surface formed at said proximal end, and a radial projection extending into said recess, the engagement of the radial projection with the recess positively securing the needle holder against distal movement within the barrel;
a finger fixed to, and extending distally from, the plunger within the barrel and alongside said cylindrical wall, the distally extending finger having a distal end engageable with said cam surface for urging said resilient finger radially inward to disengage said radial projection from said recess; and
a radial projection on one of said fingers, and a notch formed in the other of said fingers, said projection and notch being engageable with each other for latching said fingers together while the radial projection is disengaged from said recess, whereby the needle holder can be withdrawn into the barrel upon proximal movement of the plunger.
